(19) United States
(12) Patent Application Publication
(10) Pub. No.: US 2025/0230049 A1
(43) Pub. Date: Jul. 17, 2025

(54) METHOD FOR MANUFACTURING CARBON NANO MATERIALS BASED ON MACHINE LEARNING MODEL AND SYSTEM PERFORMING THE SAME

(71) Applicant: AweXome Ray, Inc., Anyang-si (KR)

(72) Inventors: Hong Soo CHOI, Seoul (KR); Keun Soo JEONG, Seoul (KR); Young Bae KIM, Anyang-si (KR); Ji Won CHOI, Anyang-si (KR); Se Hoon GIHM, Seongnam-si (KR)

(21) Appl. No.: 18/559,781

(22) PCT Filed: Sep. 25, 2023
(86) PCT No.: PCT/KR2023/014651

§ 371 Date: Dec. 29, 2023

(51) Int. Cl. C01B 32/16; G16C 20/10; G16C 20/70

(52) U.S. Cl. CPC C01B 32/16; C01B 2202/22; C01P 2002/82; C01P 2006/40; G16C 20/10; G16C 20/70

(57) ABSTRACT

Provided is a method of manufacturing a carbon nano material based on a machine learning model. The method includes obtaining first control information on a process of synthesizing carbon nano material. The method includes obtaining analysis information on the synthesized carbon nano material in real time based on the first control information. The method includes managing the first control information and the analysis information in a database. The method includes training a machine learning model using information managed in the database. The method includes synthesizing the carbon nano material by applying second control information in which the first control information for the process is adjusted based on the trained machine learning model.

### BACKGROUND

### Technical Field

Example embodiments relate to technology by which continuously mass-produce carbon nano materials with uniform physical properties based on a machine learning model.

### Description of the Related Art

The carbon nanotube (CNT), an example of a carbon nano material, is a material in which graphite surfaces are rolled up into a nano-sized diameter, and the carbon nanotube has excellent mechanical, thermal and electrical properties. For example, the tensile strength of CNTs is about 100 times that of steel, the thermal conductivity of the CNTs is similar to that of diamond, the electrical conductivity of the CNTs is approximately 1000 times that of copper. Accordingly, active research is being conducted to utilize the excellent properties of carbon nano materials such as CNTs in various industrial fields.

### BRIEF SUMMARY

The inventors of the present disclosure have acknowledged that even if the carbon nano material such as the CNTs are manufactured under the same manufacturing process conditions, their physical properties may not be maintained uniformly because various external factors affect the manufacturing process. In other words, the inventors have realized that it is difficult to continuously mass-produce carbon nano materials with uniform physical properties because the synthesized results may vary depending on various external factors and manufacturing process conditions.

The inventors have researched to improve quality prediction and yield by applying machine learning models to special manufacturing processes such as carbon nano materials. That is, the inventors have come up with a method of applying a machine learning model to the manufacturing process of carbon nano material so that carbon nano material with uniform physical properties can be continuously mass-produced.

Accordingly, an aspect provides technology by which continuously mass-produce carbon nano materials with uniform physical properties based on a machine learning model. The technical tasks to be achieved by the present example embodiments are not limited to the technical tasks described above, and other technical tasks may be inferred from the following example embodiments.

According to an aspect, there is provided a method of manufacturing a carbon nano material based on a machine learning model, the method including: obtaining first control information on a process of synthesizing carbon nano material; obtaining analysis information on the synthesized carbon nano material in real time based on the first control information; managing the first control information and the analysis information in a database; training a machine learning model using information managed in the database; and synthesizing the carbon nano material by applying second control information in which the first control information for the process is adjusted based on the trained machine learning model.

According to an example embodiment, the analysis information may include information that is analyzed in real time about the synthesized carbon nano material using a Raman spectroscopy and a camera.

According to an example embodiment, the analysis information may include information on a Raman peak intensity ratio of the synthesized carbon nano material that is analyzed in real time using the Raman spectroscopy and information that is analyzed in real time regarding a width and a thickness of the synthesized carbon nano material using the camera.

According to an example embodiment, the analysis information may further include information on electrical conductivity of the synthesized carbon nano material that is analyzed in real time.

According to an example embodiment, the carbon nano material may include a carbon nanotube (CNT).

According to an example embodiment, the carbon nanotube may be synthesized by at least one method among chemical vapor deposition (CVD), arc discharge and laser ablation.

According to an example embodiment, the first control information may include information on temperature of a heat source corresponding to a predetermined location in an electric furnace required for the process of synthesizing the carbon nano material.

According to an example embodiment, the first control information may include information on an amount and speed of input of a raw material used in the process of synthesizing the carbon nano material.

According to an example embodiment, the first control information may include information on an amount of transport gas inputted in the process of synthesizing the carbon nano material.

According to an example embodiment, the first control information may include information on speed of a roller that transports the carbon nano material synthesized in the process.

According to an example embodiment, the machine learning model may be updated based on the obtained first control information, the obtained analysis information and history information accumulated in the database.

According to an example embodiment, may be provided is a computer-readable non-transitory recording medium having a program for executing the method of manufacturing a carbon nano material based on a machine learning model, on a computer.

According to another aspect, there is provided a system that performs a method of manufacturing a carbon nano material based on a machine learning model, the system including: a communication apparatus; at least one database for managing information; and a controller that is configured to obtain first control information on a process of synthesizing carbon nano material, obtain analysis information on the synthesized carbon nano material in real time based on the first control information, instruct the first control information and the analysis information to be managed in a database, train a machine learning model using information managed in the database, and synthesize the carbon nano material by applying second control information in which the first control information for the process is adjusted based on the trained machine learning model.

According to an example embodiment, the analysis information may include information on a Raman peak intensity ratio of the synthesized carbon nano material that is analyzed in real time using the Raman spectroscopy and information that is analyzed in real time regarding a width and a thickness of the synthesized carbon nano material using the camera.

According to an example embodiment, the analysis information may further include information on electrical conductivity of the synthesized carbon nano material that is analyzed in real time.

According to an example embodiment, the carbon nano material may include a carbon nanotube that is synthesized by at least one method among chemical vapor deposition, arc discharge and laser ablation.

According to an example embodiment, the first control information may include information on temperature of a heat source corresponding to a predetermined location in an electric furnace required for the process of synthesizing the carbon nano material.

According to an example embodiment, the first control information may include information on an amount and speed of input of a raw material used in the process of synthesizing the carbon nano material.

According to an example embodiment, the first control information may include information on an amount of transport gas inputted in the process of synthesizing the carbon nano material.

According to an example embodiment, the first control information may include information on speed of a roller that transports the carbon nano material synthesized in the process.

Specific details of other example embodiments are included in the detailed description and drawings.

According to example embodiments, through a machine learning model, it is possible to analyze the relationship between various conditions by which the physical properties of carbon nano material are changed in addition to controllable condition in a process.

Further, according to example embodiments, it is possible to adjust and apply control information based on the machine learning model in order to continuously mass-produce carbon nano materials with uniform physical properties that meet reference values.

Further, according to example embodiments, efficiency may be improved by modifying the process for synthesizing carbon nano material since control information can be adjusted based on analysis information in which the carbon nano material is analyzed in real time.

Further, according to example embodiments, it is possible to continuously mass-produce carbon nano material having uniform physical properties even in various external environments and manufacturing processes through a machine learning model.

Further, according to example embodiments, the processes may be monitored and controlled remotely, allowing rapid response when an abnormality occurs, thereby improving the efficiency of the process.

The effect of the example embodiments are not limited to the above-described effects, and other effects not described would be clearly understood by those skilled in the art from the description of the claims.

### BRIEF DESCRIPTION OF THE SEVERAL VIEWS OF THE DRAWINGS

FIGS. 1A to 1C are diagrams according to an example embodiment to explain a process for synthesizing carbon nano materials.

FIG. 2 is a diagram for explaining the process of adjusting control information based on a machine learning model in the process of synthesizing carbon nano material according to an example embodiment.

FIG. 3 is a diagram for explaining environment information, control information and analysis information according to an example embodiment.

FIG. 4 shows a carbon nanotube sheet (a CNT sheet) according to an example embodiment for explaining analysis information.

FIG. 5 shows a picture of a database by analyzing a CNT film through a machine vision according to an example embodiment.

FIG. 6 is a flowchart of a method for synthesizing a carbon nano material based on a machine learning model according to an example embodiment.

FIG. 7 is a block diagram of a system performing the method of FIG. 6 according to an example embodiment.

### DETAILED DESCRIPTION

Terms used in the example embodiments are selected from currently widely used general terms when possible while considering the functions in the present disclosure. However, the terms may vary depending on the intention or precedent of a person skilled in the art, the emergence of new technology, and the like. Further, in certain cases, there are also terms arbitrarily selected by the applicant, and in the cases, the meaning will be described in detail in the corresponding descriptions. Therefore, the terms used in the present disclosure should be defined based on the meaning of the terms and the contents of the present disclosure, rather than the simple names of the terms.

Throughout the specification, when a part is described as “comprising or including” a component, it does not exclude another component but may further include another component unless otherwise stated. Furthermore, terms such as “ . . . unit,” “ . . . group,” and “ . . . module” described in the specification mean a unit that processes at least one function or operation, which may be implemented as hardware, software, or a combination thereof.

Expression “at least one of a, b and c” described throughout the specification may include “a alone,” “b alone,” “c alone,” “a and b,” “a and c,” “b and c” or “all of a, b and c.”

Hereinafter, example embodiments of the present disclosure will be described in detail with reference to the accompanying drawings so that those of ordinary skill in the art to which the present disclosure pertains may easily implement them. However, the present disclosure may be implemented in multiple different forms and is not limited to the example embodiments described herein.

Hereinafter, example embodiments will be described in detail with reference to the drawings.

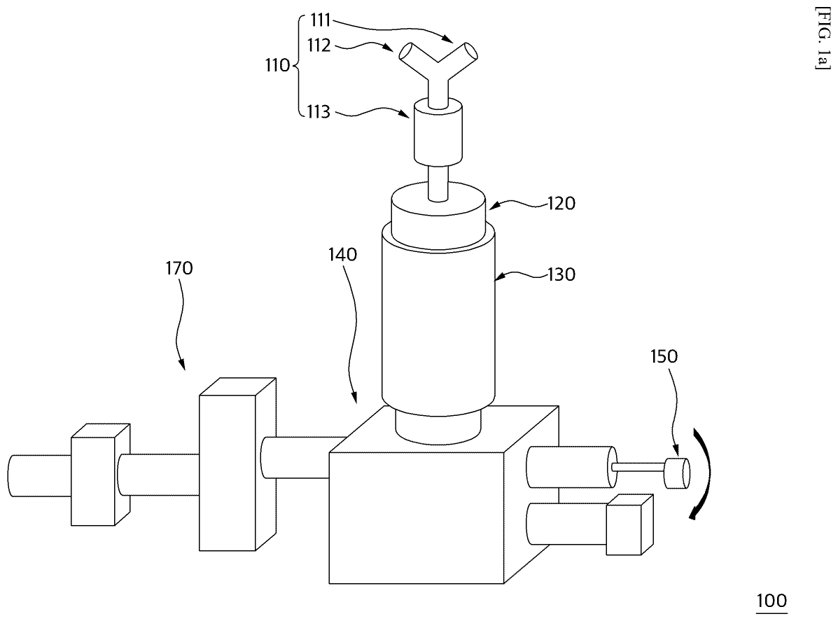

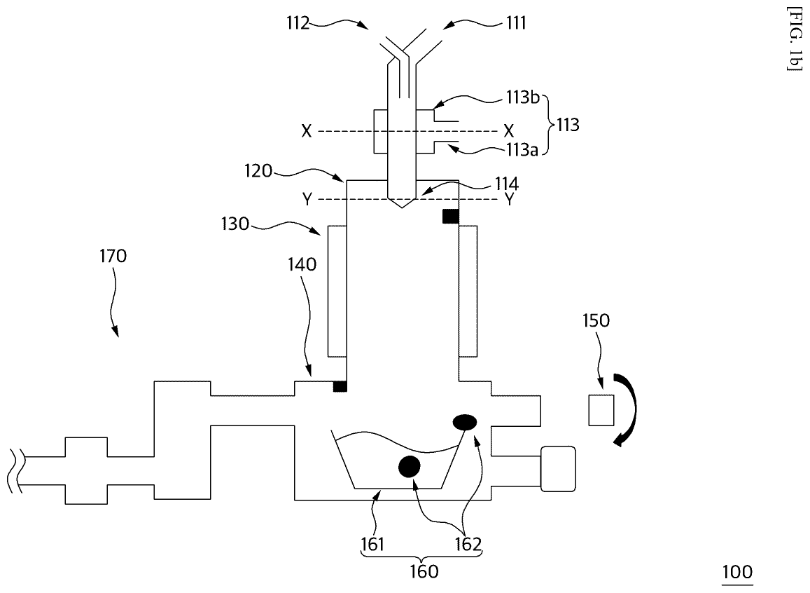

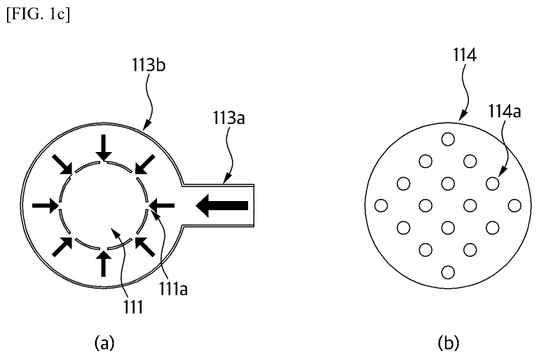

FIGS. 1A to 1C are diagrams according to an example embodiment to explain a process for synthesizing carbon nano materials.

Specifically, FIGS. 1A and 1B are a perspective view and a cross-sectional view of an apparatus for synthesizing carbon nano material according to an example embodiment, and FIG. 1C shows cross-sectional views of portions X and Y shown in FIG. 1B according to an example embodiment.

Here, as an example embodiment, the carbon nano material may include graphene, graphite and a CNT. For example, the carbon nano material may contain not only a carbon nanotube that is a short fiber and but also a carbon nanotube yarn that is a long fiber in which a plurality of CNTs are connected and a carbon nanotube sheet (a CNT sheet) processed therefrom. The carbon nanotube sheet may contain a yarn in which CNTs are aligned, and specifically, a carbon nanotube sheet with an array structure in which carbon nanotube aggregated yarns are aligned in one direction may be manufactured. The term “yarn” refers to a product formed by growing CNTs in the form of a fiber or by aggregating and/or fusing a plurality of CNTs in the form of a fiber.

FIGS. 1A to 1C illustrate an apparatus 100 for manufacturing a carbon nanotube yarn with a direct yarn manufacturing method according to an example embodiment. The direct yarn manufacturing method synthesizes CNTs at the top of an electric furnace by injecting carbon raw materials and catalysts together with transport gas into the high-temperature electric furnace, and obtains fibers by winding the continuous carbon nanotube aggregates that come down to the bottom. In the direct yarn manufacturing method, by supplying the materials necessary for the carbon nanotube synthesis to the electric furnace, continuous fiber production is possible, resulting in high production efficiency. The apparatus 100 for manufacturing a carbon nanotube yarn includes a supply part 110 that supplies materials necessary for synthesizing carbon nanotube yarns, a body part 120 provided with a space for synthesizing materials supplied through the supply part 110 into a carbon nanotube yarn, a heating part 130 that controls the temperature inside the body part 120, a space 140 formed at the lower part of the body part 120 and having a space inside, a winding part 150 that is connected to the space 140 and winds the carbon nanotube yarn synthesized in the body part 120, and a suction part 170 that sucks in the gas inside the space 140. For example, the supply part 110 may supply materials necessary for synthesizing carbon nanotube yarns into the body part 120, which will be described later. The required materials include carbon raw material, catalyst and transport gas. The supply part 110 may have an injection part through which a material is injected and a flow path through which the injected material may flow within. More specifically, the supply part 110 may include a carbon supply part 111, a catalyst supply part 112, a gas supply part 113 and a nozzle part 114. The carbon supply part 111 supplies carbon raw materials, and the catalyst supply part 112 supplies catalysts. The carbon supply part 111 and the catalyst supply part 112 are formed at the top of the apparatus 100 for manufacturing carbon nanotube yarn. The carbon supply part 111 and the catalyst supply part 112 may be formed similar to a “Y” shape. The upper part of the carbon supply part 111 is tilted to the right and from the middle part of the carbon supply part 111 may have a shape extending long downward. The carbon supply part 111 may include a flow path through which fluid can flow. The upper part of the catalyst supply part 112 is tilted to the left, and from the middle part of the catalyst supply part 112 extends long toward the bottom like the carbon supply part 111. The catalyst supply part 112 is connected to the carbon supply part 111 by being inserted into the upper part of the carbon supply part 111.

According to an example embodiment, the carbon raw material supplied through the carbon supply part 111 may be mixed with the catalyst supplied through the catalyst supply part 112 at the lower part of the carbon supply part 111. Here, the supply of catalyst needs to be appropriately adjusted based on the amount of carbon raw material supplied. This is because excess catalyst may act as an impurity contained in the synthesized carbon nanotube yarn and reduce the purity of the synthesized carbon nanotube yarn. For this, the diameter of the carbon supply part 111 may be larger than the diameter of the catalyst supply part 112. Preferably, the diameter of the carbon supply part 111 may be 2 to 50 times the diameter of the catalyst supply part 112.

According to an example embodiment, the carbon raw material supplied through the carbon supply part 111 may be a carbon compound in gaseous or liquid state. For example, the carbon raw material may be a single of known substances such as ethane, methane, ethanol, methanol and propanol, or may be a combination thereof.

According to an example embodiment, the catalyst supplied through the catalyst supply part 112 may perform the function of initiating the synthesis of carbon nanotube yarn. For example, the catalyst may be a compound such as ferrocene, cobaltocene and osmocene. In addition to the catalyst, the catalyst supply part 112 may be supplied with a catalyst activator that promotes the action of the catalyst. The carbon supply part and the catalyst supply part described above do not necessarily have to be formed as separate supply pipes. The carbon supply part and catalyst supply part may be formed as one supply pipe.

According to an example embodiment, the gas supply part 113 may supply transport gas. In an example embodiment, the gas supply part 113 is mounted in the middle part of the carbon supply part 111. Specifically, the gas supply part 113 may include a gas inlet part 113a and a circulation part 113b. One end of the gas inlet part 113a is connected to a transport gas storage (not illustrated) and an opposite end of the gas inlet part 113a is connected to the circulation part 113b. The circulation part 113b may have a cylindrical shape. The circulation part 113b is coupled to the outer peripheral surface of the middle portion of the carbon supply part 111. A plurality of injection holes 111a may be formed in a portion of the outer peripheral surface of the carbon supply part 111 coupled to the circulation part 113b (see (a) of FIG. 1C). The injection holes 111a of the carbon supply part 111 may be formed at regular intervals on the outer peripheral surface of the carbon supply part 111. The transport gas flowing into the gas inlet part 113a moves to the circulation part 113b, circulates inside the circulation part 113b, and is then injected into the carbon supply part 111 through the plurality of injection holes 111a formed on the outer peripheral surface of the carbon supply part 111. When the transport gas is mixed with carbon raw material and catalyst and supplied into the body part 120, the carbon nanotube yarn may easily move toward the bottom of the body part 120 without sticking to the inner wall of the body part 120. Therefore, the transport gas needs to be uniformly mixed with the carbon raw material and catalyst. In an example embodiment, as described above, by forming the injection holes 111a on the outer peripheral surface of the carbon supply part 111, the transport gas may uniformly injected from various directions, and thus the transport gas may be uniformly mixed with the carbon raw material and the catalyst.

In an example embodiment, only argon (Ar) gas may be used as a transport gas, only hydrogen (H₂) gas may be used as a transport gas, or argon and hydrogen gas may be mixed and used as a transport gas. Argon gas is an inert gas, and transports the synthesized carbon nanotube yarn without reacting with the carbon raw material inside the high temperature body part 120. Further, argon gas does not affect the activity of the catalyst. Hydrogen gas is a reducing gas and can contribute to synthesizing high-density carbon nanotube yarn by removing amorphous carbon. In an example embodiment, the injection ratio of hydrogen gas and argon gas may be set to 0.5:100 to 50:100. However, the mixing ratio of the transport gas is not necessarily limited thereto. Further, inert gas and reducing gas are not limited to argon gas and hydrogen gas. The inert gas may be changed to a known gas such as nitrogen gas, and the reducing gas may be changed to a known gas such as ammonia.

According to an example embodiment, a mixture of carbon raw material, catalyst and transport gas (hereinafter referred to as “mixture”) may be injected into the body part 120, which will be described later, through the nozzle part 114 of the supply part 110. The nozzle part 114 is formed in the lower part of the supply part 110 and may be located in the upper inner part of the body part 120. The nozzle part 114 may be formed in a cone shape of which diameter decreases as it goes downward. A plurality of spray holes 114a may be formed on the outer peripheral surface of the nozzle part 114 (see (b) in FIG. 1C). The mixture injected from the top of the supply part 110 reaches the nozzle part 114 and may be uniformly sprayed onto the body part 120 through the plurality of spray holes 114a formed in the nozzle part 114. Uniform spraying of the mixture may contribute to the synthesized carbon nanotube yarn having even density and uniform physical properties.

In addition to the elements of the supply part 110 described above, the supply part 110 may be equipped with a mass flow controller (MFC) and a pump, and may control the supply pressure and supply amount of the fluid flowing within the supply part 110 in real time. Further, the structure of the supply part described above is an example structure for explaining an example embodiment, and the supply part of the present disclosure is not limited thereto. The shape of the supply part or the location of the supply part connected to the body part may be changed in various ways.

According to an example embodiment, a carbon nanotube yarn is synthesized inside the body part 120. The body part 120 has a cylindrical shape extending long in the vertical direction, and includes a hollow space inside. The upper part of the body part 120 is connected to the supply part 110 described above. When the mixture is supplied into the body part 120 through the supply part 110, the mixture moves downward inside the body part 120 by gravity or the supply pressure of the mixture. The mixture is synthesized into a carbon nanotube yarn during the process. Here, the internal temperature of the body part 120 may be heated to a temperature for synthesizing carbon nanotube yarn by the heating part 130, which will be described later. The synthesized carbon nanotube yarn is discharged to the lower part of the body part 120. The body part 120 may be made of a heat-resistant material or may be formed by coating its interior with a heat-resistant material. The shape of the body part 120 described above does not limit the scope of the present disclosure. The shape of the body part 120, especially the structure of the internal space where the carbon nanotube yarn is synthesized, may be changed in various ways. For example, by forming a separate injection part in the middle of the body part 120, additives that improve the properties of the carbon nanotube yarn may be injected.

According to an example embodiment, the heating part 130 may control the internal temperature of the body part 120 to a predetermined temperature. Here, the predetermined temperature does not indicate a temperature of a fixed specific value. The synthesis speed of carbon nanotube yarn may change depending on temperature, and the synthesis speed may affect the physical properties of the carbon nanotube yarn, and thus an operator of the apparatus may take this into account and design the predetermined temperature appropriately. In an example embodiment, the electric furnace is the heating part 130, and as long as it is an apparatus that can uniformly heat the internal temperature of the body part 120 as a whole, the heating part 130 may be formed of various apparatus regardless of the heating method. For example, the heating part 130 may be formed as a gas heating type and an electric heating type.

According to an example embodiment, a carbon nanotube yarn discharged from the bottom of the body part 120 moves to the space 140. The space 140 is connected to the lower part of the body part 120, and may be formed in a box shape with a space inside. The carbon nanotube yarn and gas including transport gas may exist in the space 140.

According to an example embodiment, the carbon nanotube yarn that moved to the space 140 may be wound by the winding part 150 connected to one side of the space 140. The winding part 150 may include a motor and a winch fastened to the motor, and as the winch rotates in one direction by the motor, the carbon nanotube yarn may be wound around the winch. The rotation speed of the winch that winds the carbon nanotube yarn may be appropriately designed based on the synthesis speed of the carbon nanotube yarn.

The apparatus 100 for manufacturing carbon nanotube yarn according to an example embodiment may further include a yielding part 160 that improves the density of the carbon nanotube yarn synthesized between the body part 120 and the winding part 150. The yielding part 160 may be formed inside the space 140. The yielding part 160 may contribute to producing high-density carbon nanotube yarn by shrinking the synthesized carbon nanotube aggregate. The yielding part 160 includes a container 161 containing a solvent. Solvents such as water, acetone and dimethylformamide (DMF) may be used. The yielding part 160 may further include one or more transport rollers 162. In an example embodiment, the transport roller 162 may be formed one on each side of the lower part of the container 161 containing the solvent and the upper part of the container 161, and the transport roller 162 formed one side of the upper part may be connected to the winding part 150.

Information obtained during the process, such as the control information and the analysis information described in the present disclosure may not only be information obtained in the process shown in FIGS. 1A to 1C, but may also be information obtained in a different process.

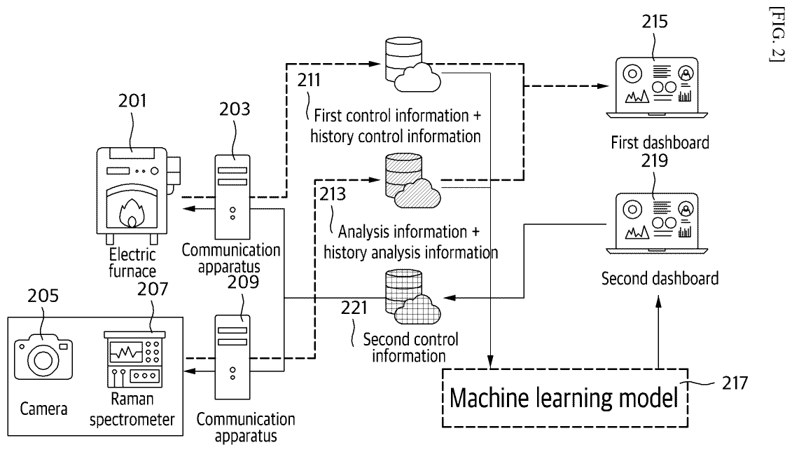

FIG. 2 is a diagram for explaining the process of adjusting control information based on a machine learning model in the process of synthesizing carbon nano material according to an example embodiment.

Referring to FIG. 2, an electric furnace 201 is a heating apparatus, and the temperature of the area heated by the electric furnace 201 may be controlled. Specifically, the area heated by the electric furnace 201 may be divided by predetermined locations, and the physical properties of the synthesized carbon nano material may vary as the temperature of the heat source is controlled for each predetermined location. In addition to the electric furnace 201, many equipment (not illustrated) may be additionally used in the process of synthesizing carbon nano material. Information related to each equipment may be transmitted and received through a communication apparatus 203 to a system that performs a method of manufacturing carbon nano material. Thus, the system may remotely monitor and control each apparatus through the communication apparatus 203.

In order to continuously mass-produce carbon nano materials with uniform physical properties, the physical properties of the synthesized carbon nano material may be analyzed in real time, and therefrom the control information may be adjusted. For a method to analyze the physical properties of carbon nano material, various methods known in the related technical field may be applied. However, among them, analysis information such as image information, Raman peak intensity ratio and electrical conductivity may be analyzed in real time during the manufacturing process. Specifically, a camera 205 may obtain image information about the synthesized carbon nano material in real time, and a Raman spectrometer 207 may obtain the Raman peak intensity ratio for the synthesized carbon nano material in real time. Further, the electrical conductivity of the synthesized carbon nano material may be analyzed and obtained in real time. However, for example, since a method to analyze physical properties such as scanning electron microscope (SEM) cannot obtain analysis information in real time, the method may be excluded from methods for analyzing the physical properties of carbon nano materials. Information analyzed in real time through each apparatus may be transmitted to the system through a communication apparatus 209.

The system may identify first control information and history control information 211, and analysis information and history analysis information 213. Here, the history control information and the history analysis information are information accumulated in the past, and are information managed in the database. The first control information may be information received through the communication apparatus 203, and the analysis information may be information received through the communication apparatus 209. In other words, the system may identify history control information and history analysis information managed in the database, and the system may identify the first control information and the analysis information received from the communication apparatuses 203 and 209. A first dashboard 215 may display the control information and the analysis information. Further, the first dashboard 215 may additionally display the history control information and the history analysis information. Therefore, an administrator who manages the system can monitor the process based on the information displayed on the first dashboard 215, as well as identify the analysis results for the synthesized carbon nano material.

The system may train a machine learning model 217 based on the history control information and the history analysis information. The system may update the machine learning model 217 based on the first control information and real-time analysis information. The machine learning model 217 may display second control information 221 through a second dashboard 219. The second control information 221 may be transmitted to related equipment through the communication apparatuses 203 and 209. Therefore, an administrator who manages the system may monitor and control the process remotely.

Here, the second control information is control information in which the first control information is adjusted based on the machine learning model 217. Specifically, if the analysis information identified in real time through the first dashboard 215 does not meet a reference value, the machine learning model 217 may output the second control information to correspond to the reference value. Here, the reference value may be set differently depending on the field to which the manufactured carbon nano material is applied. For example, the reference value corresponding to the carbon nano material applied to semiconductors and the carbon nano material applied to air conditioning apparatuses may be set differently, and the machine learning model may output the second control information based on different reference values.

The machine learning model may output the second control information based on the first control information, the history control information, the analysis information and the history analysis information. The machine learning model may be reinforced by a method in which if the physical properties (in other words, analysis information) of the carbon nano material synthesized based on the second control information satisfy a reference value, rewards are granted.

Alternatively, the machine learning model may be generated through reinforcement learning, but the machine learning model may be generated using other algorithms. For example, various machine learning algorithms, such as unsupervised learning and rule-based machine learning algorithm, may be applied.

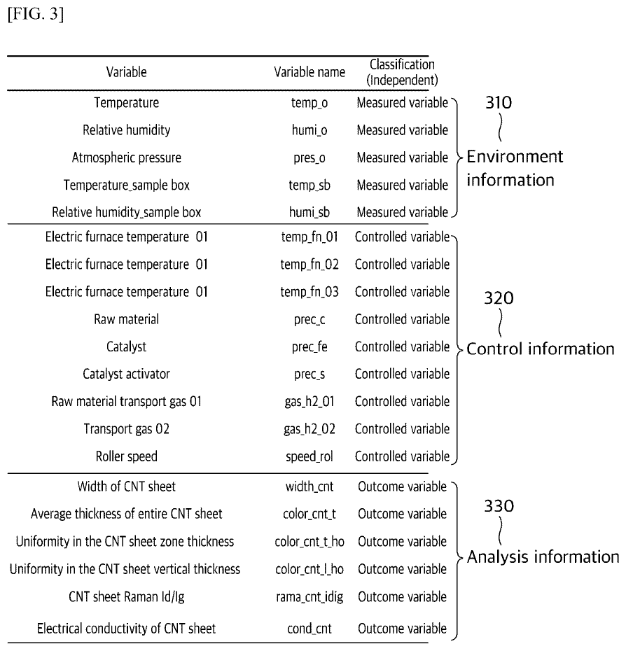

FIG. 3 is a diagram for explaining environment information, control information and analysis information according to an example embodiment.

Referring to FIG. 3, according to the example embodiment, environment information 310, control information 320 and analysis information 330 may be identified.

Here, the environment information 310 according to an example embodiment may be information related to the environment of the space where the carbon nano material is synthesized. For example, the environment information 310 may include information on temperature in the air, relative humidity in the air, atmospheric pressure, temperature and relative humidity of the yielding space inside the facility where CNTs, an example of carbon nano material, are manufactured. Specifically, the environment information 310 is information measured within a certain range related to the temperature in the air, relative humidity in the air, atmospheric pressure, temperature and relative humidity of the yielding space. Here, unlike the control information 320 which is controllable, the environment information 310 is uncontrollable information. Even under the same conditions as the control information 320, the physical properties of the carbon nano material may be changed by the environment information 310. Therefore, the machine learning model may learn the relationship between the control information and the analysis information based on the environment information.

The control information 320 according to an example embodiment is information that can be adjusted in the process of synthesizing carbon nano material, and FIG. 3 illustrates nine pieces of control information. In addition to the nine pieces of information illustrated in FIG. 3, other information may be included in the control information 320. Specifically, the electric furnace which is a heating apparatus is a long piece of equipment. The temperature of the area heated by the electric furnace may change at each predetermined location, and the physical properties of carbon nano material may be adjusted depending on the temperature. Here, the number of areas heated by the electric furnace is not limited to three as shown in FIG. 3, and the number of areas heated by the electric furnace may be determined based on various factors such as performance of the electric furnace.

Further, acetone is one example of a raw material, ferrocene is an example of a catalyst, and thiophene is an example of a catalyst activator. The amount and speed of input of the catalyst and catalyst activator may be adjusted in a relative range compared to the raw materials, and based thereon the physical properties of carbon nano material may be adjusted. Here, the catalyst activator acts as a promoter during carbon nanotube conversion and increases the carbon diffusion rate, allowing CNTs to be synthesized within a short period of time.

Further, “Raw material transport gas 01” is the gas that transports raw materials, catalysts and catalyst activators, and is adjusted in certain units within a predetermined range. “Transport gas 02” is a gas that is introduced to uniformly cause a carbon nano material synthesis reaction with uniform physical properties, and may be adjusted in certain units within a predetermined range. “Raw material transport gas 01” and “Transport gas 02” may be the same or different gases.

Further, a roller may transport a carbon nanotube sheet, which is a carbon nano material, or a film from which a carbon nanotube sheet is yielded. The physical properties of the carbon nanotube sheet may be changed by the roller speed that is adjusted in certain speed units within a predetermined speed range.

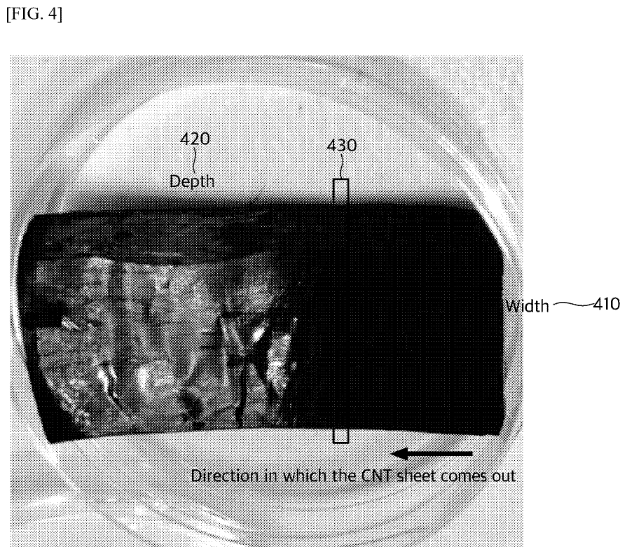

The analysis information 330 according to an example embodiment is information analyzed in real time on carbon nano material synthesized by a camera, a Raman spectrometer and other equipment. In addition, even if information is obtained by equipment that can analyze the physical properties of carbon nano material, analysis information that is not obtained in real time may be excluded from the analysis information 330. Information related to the width and the thickness of the CNT sheet may be analyzed in real time from the image information obtained through the camera. FIG. 4 shows a carbon nanotube sheet (a CNT sheet) according to the example embodiment for explaining the analysis information 330. For example, width 410 of the CNT sheet may correspond to information identified in units of a certain length within a predetermined length range based on the average value in the image information. Further, the average thickness of the entire CNT sheet may be information identified by applying a predetermined method to image information about the synthesized CNT sheet. Further, uniformity in the CNT sheet zone thickness, which indicates whether the thickness is uniform, may be information corresponding to the standard deviation of information identified by applying a predetermined method to image information. Further, unlike the uniformity in the CNT sheet zone thickness, uniformity in the CNT sheet vertical thickness may be information identified in a certain unit within a predetermined range as the thickness uniformity for an area 430 in the direction in which the CNT sheet is manufactured. Further, the Raman peak intensity ratio (Id/Ig) for a CNT sheet obtained through a Raman spectrometer may be information identified in a certain unit within a predetermined range. Further, the electrical conductivity of the carbon nanotube sheet may be information identified in real time within a predetermined range.

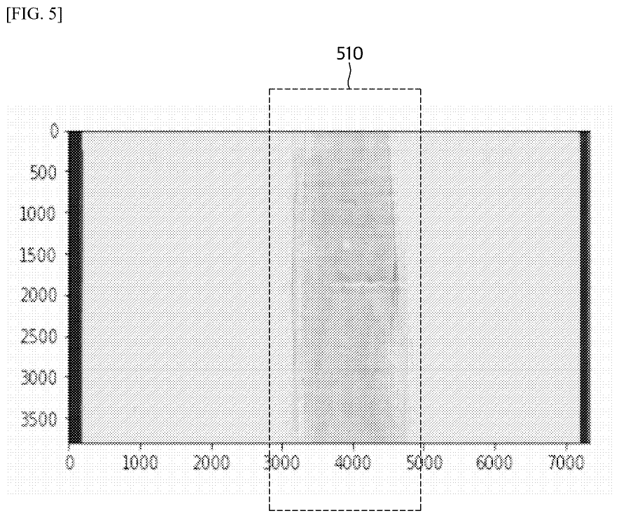

FIG. 5 shows a picture of a database by analyzing a CNT film through a machine vision according to an example embodiment.

FIG. 5 shows the result of the CNT film analysis and database generation using a machine vision camera, and an area 510 may correspond to the CNT film. Specifically, the CNT film may be photographed using the machine vision camera, and the X-axis and Y-axis of the captured image may be divided into preset pixels. For example, an image shot of the CNT film through a camera may be divided into 7200 pixels on the X-axis and 3600 pixels on the Y-axis. Here, 7200 pixels and 3600 pixels are mere example embodiments, and the present disclosure may also be applied when a CNT film is photographed using a camera with different performance. When the contrast is quantified for each pixel, it may be displayed as shown in FIG. 5.

A machine learning model may be trained based on a plurality of pieces of information databased by analyzing the CNT film using the machine vision camera. The machine learning model may analyze the physical properties of the carbon nano material captured in real time through the camera, and adjust control information so that the carbon nano material with physical properties that satisfy standard values may be generated.

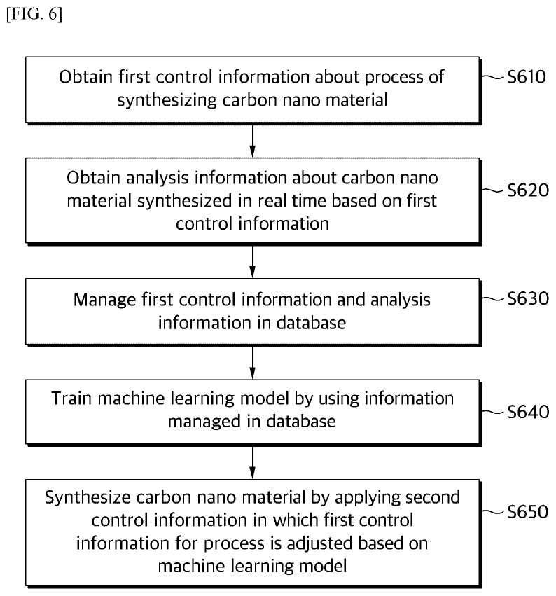

FIG. 6 is a flowchart of a method for synthesizing a carbon nano material based on a machine learning model according to an example embodiment.

Referring to FIG. 6, in operation S610, first control information about the process of synthesizing carbon nano material may be obtained.

The first control information is information that can be adjusted during the process, and the physical properties of the carbon nano material may vary accordingly. For example, the first control information may include information about the temperature of a heat source corresponding to a preset location in the electric furnace, which is a heating apparatus, in the process of synthesizing carbon nano material. For example, the first control information may include information about the amount and speed of input of raw materials used in the process of synthesizing carbon nano material. For example, the first control information may include information about the amount of transport gas that is input in the process of synthesizing carbon nano material. For example, the first control information may include information about the speed of the roller transporting the carbon nano material synthesized according to the process.

In operation S620, analysis information about the carbon nano material synthesized based on the first control information may be obtained in real time. Only real-time analyzed information (for example, image information, Raman peak intensity ratio and electrical conductivity) about the carbon nano material may be included. Information that is not analyzed in real time (for example, information on the SEM) may be excluded. Information analyzed in real time from each apparatus may be transmitted to the system through a communication apparatus.

Specifically, the analysis information may include information on real-time analysis of the Raman peak intensity ratio of the carbon nano material synthesized by using a Raman spectroscope (that is, the Raman spectrometer), and information analyzed in real time regarding the width and thickness of the carbon nano material synthesized by using a camera. Further, the analysis information may further include information on the real-time analysis of the electrical conductivity of the synthesized carbon nano material.

Here, the carbon nano material includes carbon nanotubes, and the carbon nanotubes may be synthesized by at least one method among chemical vapor deposition (CVD), arc discharge and laser ablation. What is known in the related technical field regarding the chemical vapor deposition, the arc discharge and the laser ablation may also be applied. For example, the chemical vapor deposition is a method of forming a catalyst metal film on a substrate and then etching the catalyst metal film with etching gas, and is a method of synthesizing the CNTs by reacting multiple catalyst particles with raw materials in the formed state. For example, the arc discharge is a method in which an arc discharge is generated between two carbon electrodes using a pure carbon electrode as the cathode and a metal-added carbon electrode as the anode, carbon evaporated from the anode moves to the cathode surface, and CNTs synthesized on the cathode surface are synthesized. For example, the laser ablation is a method of synthesizing CNTs by vaporizing graphite by irradiating a laser in a high temperature state filled with an inert gas. In addition, synthetic methods known in the field of technology for manufacturing carbon nano material may also be applied.

In operation S630, the first control information and the analysis information obtained in real time may be managed in a database. In operation S640, a machine learning model may be trained by using information managed in the database. The machine learning model may be updated based on the first control information, the analysis information analyzed in real time, and historical information accumulated in the database. In operation S650, the carbon nano material may be synthesized by applying second control information in which the first control information for the process is adjusted based on the machine learning model.

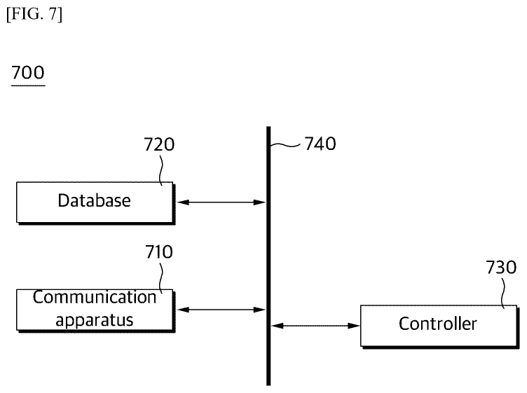

FIG. 7 is a block diagram of a system performing the method of FIG. 6 according to an example embodiment. FIG. 7 illustrates elements related to the example embodiments, but the present disclosure is not limited thereto. In addition to the elements shown in FIG. 7, other general-purpose elements may be further included. Contents that overlap the above description may be omitted.

A system 700 of FIG. 7 may be an apparatus that performs the methods described above. Referring to FIG. 7, the system 700 includes a communication apparatus 710, a database 720, a controller 730 and a bus 740. The database 720 and the controller 730 may communicate with each other through the bus 740. Each of the database 720 and the controller 730 may indicate a unit that processes at least one function or operation. Each of the database 720 and the controller 730 may be implemented as hardware, software or a combination of hardware and software.

In an example embodiment, the system 700 may include the database 720 that stores various information related to the system 700. For example, at least one instruction for operating the system 700 may be stored in the database 720. In this case, the database 720 and the controller 730 may perform various operations based on the instructions. The controller 730 may perform the above-described operations as instructions stored in the database 720 are executed in the controller 730. The database 720 may be volatile memory or non-volatile memory. For example, the database 720 may store data processed by the controller 730 and data to be processed. The database 720 may include random access memory (RAM), such as dynamic random access memory (DRAM), static random access memory (SRAM), read-only memory (ROM), electrically erasable programmable read-only memory (EEPROM), CD-ROM, Blu-ray or other optical disk storage, hard disk drive (HDD), solid state drive (SSD) or flash memory.

In an example embodiment, instructions are executed in the controller 730, the controller 730 identifies control information about the process of synthesizing carbon nano material, the controller 730 identifies analysis information about the synthesized carbon nano material in real time based on the control information, the controller 730 instructs the control information and the analysis information to be managed in the database, the controller 730 trains a machine learning model by using information managed in the database, and the controller 730 instructs adjustment of the control information related to the synthesis of the carbon nano material based on the machine learning model.

The system and various apparatuses according to the above-described example embodiments may include a controller, a memory for storing and executing program data, a permanent storage such as a disk drive, and/or a user interface device such as a communication port, a touch panel, a key and/or a button that communicates with an external device. Methods implemented as software modules or algorithms may be stored in a computer-readable recording medium as computer-readable codes or program instructions executable on the controller. Here, the computer-readable recording medium includes a magnetic storage medium (for example, ROMs, RAMs, floppy disks and hard disks) and an optically readable medium (for example, CD-ROMs and DVDs). The computer-readable recording medium may be distributed among network-connected computer systems, so that the computer-readable codes may be stored and executed in a distributed manner. The medium may be readable by a computer, stored in a memory, and executed on the controller.

The example embodiments may be represented by functional block elements and various processing steps. The functional blocks may be implemented in any number of hardware and/or software configurations that perform specific functions. For example, an example embodiment may adopt integrated circuit configurations, such as memory, processing, logic and/or look-up table, that may execute various functions by the control of one or more microprocessors or other control devices. Similar to that elements may be implemented as software programming or software elements, the example embodiments may be implemented in a programming or scripting language such as C, C++, C#, python, Java, assembler, etc., including various algorithms implemented as a combination of data structures, processes, routines, or other programming constructs. Functional aspects may be implemented in an algorithm running on one or more processors.

The above-described example embodiments are merely examples, and other embodiments may be implemented within the scope of the claims to be described later.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of manufacturing a carbon nano material based on a machine learning model, the method comprising:
obtaining first control information on a process of synthesizing carbon nano material;
obtaining analysis information on the synthesized carbon nano material in real time based on the first control information;
managing the first control information and the analysis information in a database;
training a machine learning model using information managed in the database; and
synthesizing the carbon nano material by applying second control information in which the first control information for the process is adjusted based on the trained machine learning model.

2. The method of claim 1, wherein the analysis information includes information that is analyzed in real time about the synthesized carbon nano material using a Raman spectroscopy and a camera.

3. The method of claim 2, wherein the analysis information includes information on a Raman peak intensity ratio of the synthesized carbon nano material that is analyzed in real time using the Raman spectroscopy and information that is analyzed in real time regarding a width and a thickness of the synthesized carbon nano material using the camera.

4. The method of claim 3, wherein the analysis information further includes information on electrical conductivity of the synthesized carbon nano material that is analyzed in real time.

5. The method of claim 1, wherein the carbon nano material includes a carbon nanotube (CNT).

6. The method of claim 5, wherein the carbon nanotube is synthesized by at least one method among chemical vapor deposition (CVD), arc discharge and laser ablation.

7. The method of claim 5, wherein the first control information includes information on temperature of a heat source corresponding to a predetermined location in an electric furnace required for the process of synthesizing the carbon nano material.

8. The method of claim 5, wherein the first control information includes information on an amount and speed of input of a raw material used in the process of synthesizing the carbon nano material.

9. The method of claim 5, wherein the first control information includes information on an amount of transport gas inputted in the process of synthesizing the carbon nano material.

10. The method of claim 5, wherein the first control information includes information on speed of a roller that transports the carbon nano material synthesized in the process.

11. The method of claim 1, wherein the machine learning model is updated based on the obtained first control information, the obtained analysis information and history information accumulated in the database.

12. A non-transitory computer-readable recording medium storing instructions that, when executed by one or more processors in a computing system, cause the one or more processors to perform actions, the actions comprising;
obtaining first control information on a process synthesizing carbon nano material;
obtaining analysis information on the synthesized carbon nano material in real time based on the first control information;
managing the first control information and the analysis information in a database;
training a machine model using formation managed in the database; and
synthesizing the carbon nano material by applying second control information in which the first control information for the process is adjusted based on the trained machine learning model.

13. A system that performs a method of manufacturing a carbon nano material based on a machine learning model, the system comprising:
a communication apparatus;
at least one database for managing information; and
a controller that is configured to obtain first control information on a process of synthesizing carbon nano material, obtain analysis information on the synthesized carbon nano material in real time based on the first control information, instruct the first control information and the analysis information to be managed in a database, train a machine learning model using information managed in the database, and synthesize the carbon nano material by applying second control information in which the first control information for the process is adjusted based on the trained machine learning model.

14. The system of claim 13, wherein the analysis information includes information on a Raman peak intensity ratio of the synthesized carbon nano material that is analyzed in real time using the Raman spectroscopy and information that is analyzed in real time regarding a width and a thickness of the synthesized carbon nano material using the camera.

15. The system of claim 14, wherein the analysis information further includes information on electrical conductivity of the synthesized carbon nano material that is analyzed in real time.

16. The system of claim 13, wherein the carbon nano material includes a carbon nanotube that is synthesized by at least one method among chemical vapor deposition, arc discharge and laser ablation.

17. The system of claim 16, wherein the first control information includes information on temperature of a heat source corresponding to a predetermined location in an electric furnace required for the process of synthesizing the carbon nano material.

18. The system of claim 16, wherein the first control information includes information on an amount and speed of input of a raw material used in the process of synthesizing the carbon nano material.

19. The system of claim 16, wherein the first control information includes information on an amount of transport gas inputted in the process of synthesizing the carbon nano material.

20. The system of claim 16, wherein the first control information includes information on speed of a roller that transports the carbon nano material synthesized in the process.
